(19) United States
(12) Patent Application Publication
(10) Pub. No.: US 2025/0277788 A1
(43) Pub. Date: Sep. 4, 2025

(54) SARS-COV-2 N PROTEIN-SPECIFIC SINGLE-CHAIN ANTIBODY, AND FUSION PROTEIN AND USE THEREOF

(71) Applicant: Guizhou Medical University, Anshun (CN)

(72) Inventors: Zuquan HU, Anshun (CN); Zhu ZENG, Anshun (CN); Yuxue YAN, Anshun (CN); Guofu SHANG, Anshun (CN); Liang HONG, Anshun (CN); Jianman WANG, Anshun (CN)

(73) Assignee: Guizhou Medical University, Anshun (CN)

(21) Appl. No.: 18/824,982

(22) Filed: Sep. 5, 2024

(30) Foreign Application Priority Data
Mar. 1, 2024 (CN) 202410238746.X

(51) Int. Cl. G01N 33/569; C07K 16/10; C12N 9/16; G01N 33/543

(52) U.S. Cl. CPC G01N 33/56983; C07K 16/1003; C12N 9/16; G01N 33/543; C07K 2317/622; C07K 2319/00; C12Y 301/03001; G01N 2333/165

(57) ABSTRACT

A SARS-COV-2 N protein-specific single-chain antibody, a fusion protein, and use thereof are provided. The antibody includes a heavy chain variable region V_{H}, a linker and a light chain variable region V_{L} which are connected in sequence; the amino acid sequence of the heavy chain variable region V_{H} is set forth in SEQ ID NO: 4; and the amino acid sequence of the light chain variable region V_{L} is set forth in SEQ ID NO: 6. Compared with the conventional ELISA detection, the fusion protein can greatly shorten the detection time, can catalyze the substrate to generate a color reaction which can be recognized by naked eyes, and can perform detection without special instruments.

### CROSS REFERENCE TO THE RELATED APPLICATIONS

This application is based upon and claims priority to Chinese Patent Application No. 202410238746.X, filed on Mar. 1, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC214_SequenceListing.xml, created on Aug. 19, 2024, and is 32,954 bytes in size.

### TECHNICAL FIELD

The present invention relates to the technical field of biological detection, and in particular, to a SARS-COV-2 N protein-specific single-chain antibody, and a fusion protein and use thereof.

### BACKGROUND

The emergence of severe acute respiratory syndrome coronavirus 2 (SARS-COV-2) marks the third highly pathogenic coronavirus to spill over into the human population. SARS-COV-2 can infect humans or animals through respiratory tract, contact, and aerosol transmission, and poses a serious threat to the human body. Although preventive measures such as vaccination and proper hygiene measures prevent most strains from infecting humans, the virus cannot be completely controlled due to the emergence of variants. Novel coronavirus variants Alpha, Beta, Gamma, Delta, and Omicron continue to appear and have obvious persistence. Compared with other variants, the Omicron is one with the most variations, the high transmissibility and immune escape ability of the Omicron have attracted global attention, and the Omicron has replaced the previous strain Delta as the main variant in many regions. Meanwhile, due to changes in the cell tropism, the Omicron is less pathogenic, but shows significant resistance to the neutralizing activity of vaccines, convalescent serum, and most antibody therapies. In the long term, the protection provided by antibody vaccines in the human body weakens over time. Therefore, continuous long-term monitoring of SARS-COV-2 is still needed to prevent it from causing irreversible harm to humans again.

SARS-COV-2 belongs to coronavirus genus of coronavirus family, and is mainly composed of nucleocapsid protein (N), membrane protein (M), envelope protein (E) and spike protein(S), wherein N protein has strong immunogenicity due to high sequence conservation, and can be used as a good detection marker. The current method for detecting SARS-COV-2 mainly uses reverse transcription-polymerase chain reaction (RT-PCR) as the gold standard and enzyme-linked immunosorbent assay (ELISA) as auxiliary detection. The RT-PCR detection has certain errors from sample collection to result output, which easily causes false positive, and detection work in remote areas is difficult to perform due to the requirements on technical personnel and professional equipment.

Therefore, how to provide a novel coronavirus detection method that is convenient to detect and has good sensitivity and specificity is an issue to be solved urgently by those skilled in the art.

### SUMMARY

An objective of the present invention is to provide a SARS-COV-2 N protein-specific single-chain antibody gene. Hybridoma cells are prepared by immunizing mice with a recombined SARS-COV-2 N protein, a single-chain antibody gene library is constructed by using molecular cloning methods and techniques, screening is performed by phage display technology, identifying is performed by phage ELISA and soluble scFv-ELISA, sequence determination is performed, and finally a high-affinity anti-SARS-COV-2 N protein-specific single-chain antibody and an encoding gene thereof is obtained, named H6. The gene consists of 753 nucleotides and has a sequence set forth in SEQ ID NO: 1. A heavy chain variable region V_{H} consists of 90 nucleotides (comprising three variable regions CDR H1, CDR H2, CDR H3) and has a sequence set forth in SEQ ID NO: 3. A light chain variable region V_{L} consists of 81 nucleotides (comprising three variable regions CDR L1, CDR L2, CDR L3) and has a sequence set forth in SEQ ID NO: 5.

Another objective of the present invention is to provide a SARS-COV-2 N protein-specific single-chain antibody that has a sequence set forth in SEQ ID NO: 2 and consists of 251 amino acids. The antibody mainly consists of a heavy chain variable region V_{H}, a light chain variable region V_{L} and a linker, wherein the V_{H} and the V_{L} are connected through the linker (Gly₄Ser)₃ to jointly complete the recognition and the binding of SARS-COV-2 N protein. The heavy chain variable region V_{H} of the antibody consists of 30 amino acids (comprising three variable regions CDR H1, CDR H2, CDR H3) and has a sequence set forth in SEQ ID NO: 4, and the light chain variable region V_{L} of the antibody consists of 27 amino acids (comprising three variable regions CDR L1, CDR L2, CDR L3) and has a sequence set forth in SEQ ID NO: 6.

Another objective of the present invention is to provide a fusion protein gene of a SARS-COV-2 N protein-specific single-chain antibody H6 and an AP, a sequence of which is set forth in SEQ ID NO: 7.

Another objective of the present invention is to provide a fusion protein of a SARS-COV-2 N protein-specific single-chain antibody H6 and an AP, an amino acid sequence of which is set forth in SEQ ID NO: 8. A single-chain antibody gene H6 is constructed into a pDAP2/S vector containing an AP gene to obtain an H6-AP fusion protein expression vector, and the H6-AP fusion protein can be directly applied to the detection of SARS-COV-2 after mass expression and purification are performed in E. coli.

The final objective of the present invention is to provide use of the fusion protein H6-AP of the SARS-COV-2 N protein-specific single-chain antibody in the detection of SARS-COV-2. The present invention obtains a SARS-COV-2 N protein-specific single-chain antibody with high affinity and a coding gene thereof by screening through the phage display technology, constructs a fusion protein expression vector with an alkaline phosphatase (AP), performs soluble expression in E. coli, is used to detect the spread infection condition of SARS-COV-2 in people, and provides a method for detecting SARS-COV-2 with high efficiency and low cost.

To achieve the above objective, the present invention provides the following technical solutions.

The present invention provides a SARS-COV-2 N protein-specific single-chain antibody. The antibody comprises a heavy chain variable region V_{H}, a linker and a light chain variable region V_{L} which are connected in sequence; an amino acid sequence of the heavy chain variable region V_{H} is set forth in SEQ ID NO: 4; and an amino acid sequence of the light chain variable region V_{L} is set forth in SEQ ID NO: 6.

Preferably, the linker is (Gly₄Ser)₃.

Preferably, an amino acid sequence of the antibody is set forth in SEQ ID NO: 2.

The present invention further provides a fusion protein for detecting SARS-COV-2, which comprises the antibody and an alkaline phosphatase.

Preferably, an amino acid sequence of the fusion protein is set forth in SEQ ID NO: 8.

The present invention further provides use of the fusion protein in preparing a novel coronavirus detection kit.

Preferably, the kit comprises the fusion protein and an auxiliary detection reagent.

Preferably, the auxiliary detection reagent comprises: a transparent ELISA plate, a washing buffer, a blocking solution, a color developing solution and a positive control substance. The present invention further provides a nucleic acid encoding the fusion protein.

The present invention further provides a host cell expressing the fusion protein or comprising the nucleic acid.

Compared with the prior art, the present invention has the following beneficial effects:

The present invention greatly reduces the detection limit of the novel coronavirus by constructing the fusion protein of the SARS-COV-2 N protein-specific single-chain antibody and the alkaline phosphatase. Meanwhile, the present invention can adapt to different detection environments, does not require complex instruments and equipment, and is favorable for detecting the infection of the novel coronavirus in time.

The H6-AP fusion protein prepared by the present invention can be soluble and expressed in E. coli, does not require expensive instruments and equipment and complicated operation, has low cost and is suitable for large-scale production. Finally, the detection ability of the fusion protein to SARS-COV-2 is verified by methods such as Western Blot and ELISA. Compared with the conventional ELISA detection, the fusion protein can greatly shorten the detection time, can catalyze the substrate to produce an eye-readable biosensor reading for use in devices without readouts, and can perform the detection without special instruments.

### BRIEF DESCRIPTION OF THE DRAWINGS

FIG. 1 is a technical route diagram of the present invention.

FIGS. 2A-2B are gel electropherograms of V_{H} (FIG. 2A) and V_{L} (FIG. 2B) fragments of Example 3; wherein 1-5 are V_{H} (FIG. 2A) and V_{L} (FIG. 2B) amplified from 5 hybridoma cells; and M is a DNA molecular weight standard.

FIG. 3 is a photograph of scFv fragments of Example 3; wherein 1-6 are scFvs connected with five V_{H}s and V_{L}s and one scFv connected with a mixed V_{H} and V_{L}; and M is a DNA molecular weight standard.

FIG. 4 shows the pHENHi vector structure of Example 4.

FIG. 5 shows the identification of positive clones of the antibody gene library in Example 6; wherein M is a DNA molecular weight standard.

FIG. 6 is a result diagram of a panning antibody library of a recombinant SARS-COV-2 N protein identified by phage ELISA in Example 8; wherein * marks are 10 sequenced monoclones.

FIG. 7 is a result diagram of SDS-PAGE electrophoresis detection of purified H6 antibody in Example 10; wherein M is a protein molecular weight standard.

FIG. 8 is a result diagram of the affinity detection of H6 and recombinant SARS-COV-2 N protein by surface plasmon resonance (SPR) in Example 11.

FIG. 9 is a result diagram of SDS-PAGE electrophoresis detection of purified H6-AP fusion protein in Example 12; wherein M is a protein molecular weight standard.

FIG. 10 is a graph showing the change in signal intensity of H6-AP fusion protein over time by ELISA analysis in Example 13.

FIGS. 11A-11B are graphs showing the detection limit of H6-AP fusion protein analyzed by ELISA analysis in Example 14.

FIG. 12 is a graph showing the minimal dosage of H6-AP fusion protein by ELISA analysis in Example 15.

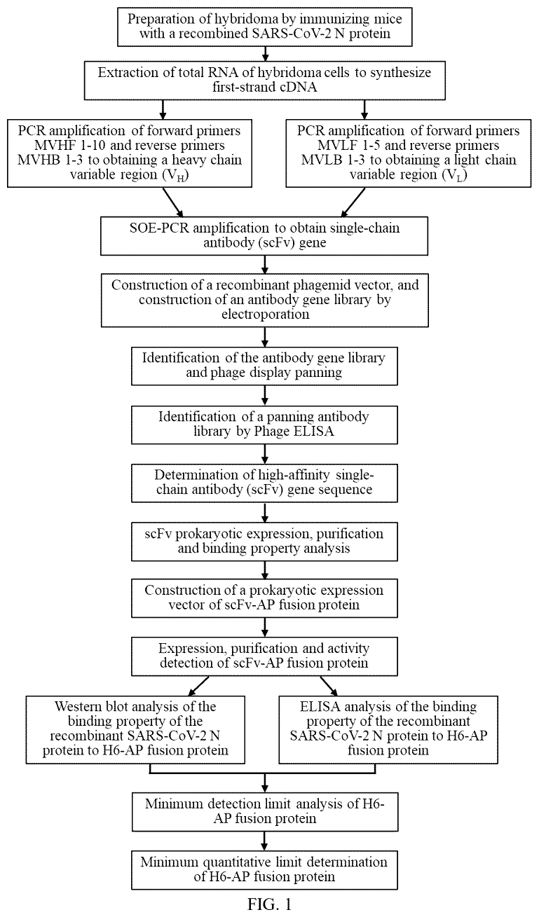

### DETAILED DESCRIPTION OF THE EMBODIMENTS

The technical solutions provided by the present invention will be described in detail below with reference to examples, which, however, should not be construed as limiting the scope of the present invention.

### Example 1: Immunization of Mice with Recombinant SARS-COV-2 N Protein to Prepare Hybridoma Cells

1) BALB/c mice were immunized with the recombinant SARS-COV-2 N protein (SEQ ID NO: YP_009724397.2) by multiple subcutaneous injections, with 25 μg of antigen per mouse each time.
2) For the first immunization, Freund's complete adjuvant was mixed with an equal volume of antigen and fully emulsified. For the subsequent two immunizations, Freund's incomplete adjuvant was mixed with an equal volume of antigen and fully emulsified. The interval between the first and second immunizations was 18 days, and the interval between the second and third immunizations was 15 days.
3) On the day 10 after 3 immunizations, the tail vein blood of the mice was collected, the antiserum titer was detected by indirect ELISA, and the mice with higher titer were selected for cell fusion.
4) Splenic lymphocytes were separated, and the separated spleen cells were fused with pre-revived SP2/0 myeloma cells at a ratio of 2:1 to 5:1 using PEG1500. The cell culture supernatant was aspirated, and positive hybridoma cells were screened by indirect ELISA. Subcloning was performed using limiting dilution until the positive rate of all cloned cell wells was 100%, which determined that a positive hybridoma cell strain has been obtained.

### Example 2: Extraction of Total RNA from Hybridoma Cells

1) The hybridoma cells of Example 1 were collected, washed three times with PBS, resuspended in 1 mL of Trizol, mixed by pipetting, and preserved at −80° C. overnight.
2) 200 μL of chloroform was added, shaken vigorously for 15 s, and placed on ice for 5 min.
3) The mixture was centrifuged at 4° C. and 12000 r/min for 10 min, the supernatant was transferred to a new RNase-free EP tube, an equal volume of isopropanol was added, and the mixture was gently inverted to mix and allowed to stand at room temperature for 5 min.
4) The mixture was centrifuged at 4° C. and 12000 r/min for 15 min, the supernatant was discarded, and the precipitate was washed with 1 mL of 75% (v/v) ethanol prepared with DEPC water.
5) The mixture was centrifuged at 4° C. and 7500 r/min for 5 min, and the supernatant was discarded, which were repeated once; the supernatant was aspirated and allowed to stand until the ethanol evaporated, and 20 μL of DEPC water was added for dissolution.
6) First-strand cDNA was synthesized by reverse transcription using the RNA obtained in step 5 as a template using a Thermo Scientific™ reverse transcription kit (purchased from Thermo Fisher, and operated according to the instructions of the kit) and a specific primer Oligo (dT)₁₈.

### Example 3: PCR Amplification of Heavy Chain Variable Region (V_{H}), light chain variable region (V_{L}) and Single-Chain Antibody (scFv) Genes

1) PCR amplification of V_{H} and V_{L} genes

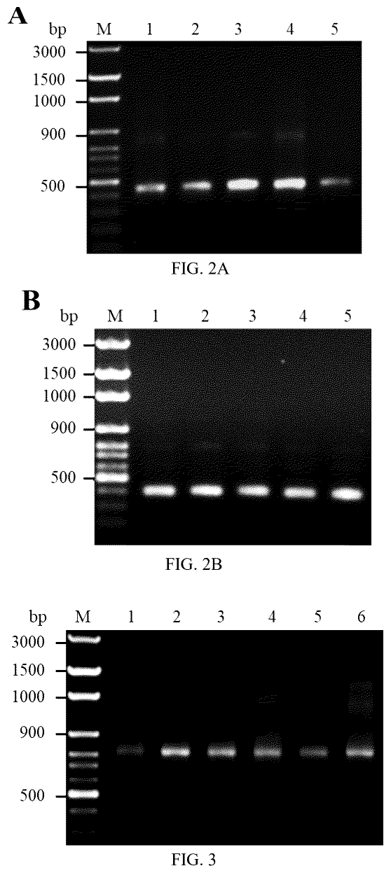

The cDNA synthesized by reverse transcription in Example 2 was used as a template, and the MVHF1-10 and reverse primers MVHB1-3 of the heavy chain variable region forward primers were used to perform PCR amplification to synthesize the V_{H} fragment; and the forward primers MVLF1-5 and reverse primers MVLB1-3 of the light chain variable region were used to perform PCR amplification to synthesize the V_{L} fragment (the primer sequences are shown in Table 4, and the V_{H} and V_{L} fragments are shown in FIGS. 2A-2B) (the linker was introduced by primers MVHB1-3 and MVLF1-5).

The heavy chain variable region fragment (V_{H}) amplification reaction system is as follows:

TABLE 1
V_{H} amplification reaction system

| | |
|---|---|
| 10× buffer II | 2.5 μL |
| dNTP Mixture | 2 μL |
| MVHF1-10 | 1 μL |
| MVHB1-3 | 1 μL |
| cDNA | 1 μL |
| LA Taq | 0.25 μL |
| Supplemented with ddH₂O to | 50 μL |

Mixing the solution and immediately performing centrifugation;
The light chain variable region fragment (V_{L}) amplification reaction system is as follows:

TABLE 2
V_{L} amplification reaction system

| | |
|---|---|
| 10× buffer II | 2.5 μL |
| dNTP Mixture | 2 μL |
| MVLF1-5 | 1 μL |
| MVLB1-3 | 1 μL |
| cDNA | 1 μL |
| LA Taq | 0.25 μL |
| Supplemented with ddH₂O to | 50 μL |

Mixing the solution and immediately performing centrifugation;

PCR reaction procedures:

TABLE 3
PCR reaction procedures

| | |
|---|---|
| 95° C. | 5 min |
| 94° C. | 1 min |
| 55° C. | 1 min |
| 72° C. | 80 s |
| 72° C. | 10 min |
| 12° C. | 10 min |

TABLE 4
PCR amplification primer sequences
for single-chain antibody

| SEQ | | | Primer |
|---|---|---|---|
| ID | Primer | | fragment |
| NO: | name | Primer sequence (5′→3′) | size (bp) |
| 9 | MVHF1 | CATGCCATGACTCGCGGCCCAGCCGGCCATGGCCG | 58 |
| | | AKGTRCAGCTTCAGGAGTCRGGA | |
| 10 | MVHF2 | CATGCCATGACTCGCGGCCCAGCCGGCCATGGCCC | 58 |
| | | AGGTGCAGCTGAAGSAGTCWGGM | |
| 11 | MVHF3 | CATGCCATGACTCGCGGCCCAGCCGGCCATGGCCSA | 58 |
| | | GGTYCAGCTGCARCAGTCWGGD | |
| 12 | MVHF4 | CATGCCATGACTCGCGGCCCAGCCGGCCATGGCCSA | 58 |
| | | GGTCCARCTGCAGSARYCTGGR | |
| 13 | MVHF5 | CATGCCATGACTCGCGGCCCAGCCGGCCATGGCCG | 58 |
| | | AGGTTCAGCTGCAGCAGTCTGGG | |
| 14 | MVHF6 | CATGCCATGACTCGCGGCCCAGCCGGCCATGGCCG | 58 |
| | | ARGTGAAGCTGGTGGARTCTGGR | |
| 15 | MVHF7 | CATGCCATGACTCGCGGCCCAGCCGGCCATGGCCG | 58 |
| | | AGGTGAAGCTTCTCGAGTCTGGA | |
| 16 | MVHF8 | CATGCCATGACTCGCGGCCCAGCCGGCCATGGCCG | 57 |
| | | ARGTGAAGCTKGAKGAGWCTGR | |
| 17 | MVHF9 | CATGCCATGACTCGCGGCCCAGCCGGCCATGGCCGA | 58 |
| | | VGTGMWGCTKGTGGAGTCTGGK | |
| 18 | MVHF10 | CATGCCATGACTCGCGGCCCAGCCGGCCATGGCCSA | 58 |
| | | GGTYCAGCTKCAGCAGTCTGGA | |
| 19 | MVHB1 | TCCAGAACCGCCACCGCCGCTACCGCCGCCACCGA | 57 |
| | | CAGRTGGGGSTGTYGTTTTGGC | |
| 20 | MVHB2 | TCCAGAACCGCCACCGCCGCTACCGCCGCCACCGA | 54 |
| | | CAGATGGGGCTGTTGTTKT | |
| 21 | MVHB3 | TCCAGAACCGCCACCGCCGCTACCGCCGCCACCGA | 57 |
| | | CATTTGGGAAGGACTGACTCTC | |
| 22 | MVLF1 | AGCGGCGGTGGCGGTTCTGGAGGCGGCGGTTCTGA | 53 |
| | | CATTGTGMTGWCACAGTC | |
| 23 | MVLF2 | AGCGGCGGTGGCGGTTCTGGAGGCGGCGGTTCTG | 53 |
| | | ATRTTKTGATGACCCARAC | |
| 24 | MVLF3 | AGCGGCGGTGGCGGTTCTGGAGGCGGCGGTTCTRA | 53 |
| | | MATTGTGMTGACCCAATC | |
| 25 | MVLF4 | AGCGGCGGTGGCGGTTCTGGAGGCGGCGGTTCTSA | 53 |
| | | AAWTGTKCTSACCCAGTC | |
| 26 | MVLF5 | AGCGGCGGTGGCGGTTCTGGAGGCGGCGGTTCTGA | 53 |
| | | YATYCAGATGACMCAGWC | |
| 27 | MVLB1 | CTAGTGGTACTCCACGCGGCCGCGTCGACAGCMCG | 50 |
| | | TTTCAGYTCCARYTT | |
| 28 | MVLB2 | CTAGTGGTACTCCACGCGGCCGCGTCGACAGCMCG | 50 |
| | | TTTKATYTCCARYTT | |
| 29 | MVLB3 | CTAGTGGTACTCCACGCGGCCGCGTCGACAGCMCG | 53 |
| | | TTTBAKYTCTATCTTTGT | |

Note:
* R = A or G; Y = C or T; M = C or A; K = G or T; W = A or T; S = C or G; D = G, A or T; V = A, C or G. These sequences are set forth in SEQ ID NOS: 9 to 29.

2) The V_{H} and V_{L} fragments amplified in step 1) were purified by a DNA gel recovery kit (purchased from TIANGEN, and operated according to the instructions of the kit).
3) Amplification of scFv gene by SOE-PCR

Equimolar V_{H} and V_{L} fragments were added as templates, and SOE-PCR amplification was performed using forward primers MVHF1-10 and reverse primers MVLB1-3 to obtain the scFv gene (as shown in FIG. 3).

The SOE-PCR was completed in two steps. The first step of PCR reaction system was as follows: 2× GC buffer I, 25 μL; dNTP Mixture, 4 μL; MVHF1-10, 2 μL; MVLB1-3, 2 μL; V_{H}, 200 ng; V_{L}, 200 ng; LA Taq, 0.5 μL; supplemented with ddH₂O to 50 μL.

The first step of PCR reaction: 7 cycles (95° C., 5 min; 55° C., 2 min; 72° C., 15 min); 12° C. for 10 min.

The second step of PCR reaction system were as follows: 2×GC buffer I, 25 μL; dNTP Mixture, 4 μL; MVHF1-10, 2 μL; MVLB1-3, 2 μL; DNA (first-step PCR product), 1 μL; LA Taq, 0.5 μL; supplemented with ddH₂O to 50 μL. The second step of PCR reaction: 30 cycles (95° C., 5 min; 94° C., 1 min; 55° C., 80 s; 72° C., 2 min; 72° C., 10 min); 12° C. for 10 min.
4) The scFv fragment obtained in step 3 was recovered and purified by using a DNA gel recovery kit (purchased from TIANGEN, and operated according to the instructions of the kit).

### Example 4: Enzyme Digestion and Ligation of pHENHi Vector and scFv Fragment

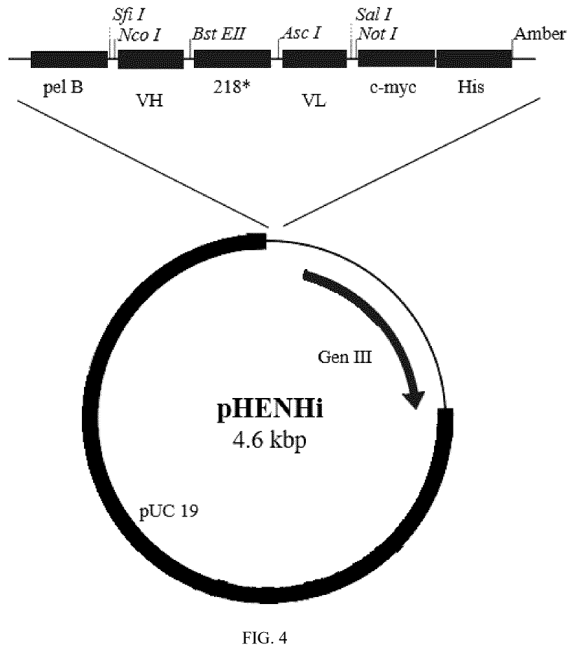

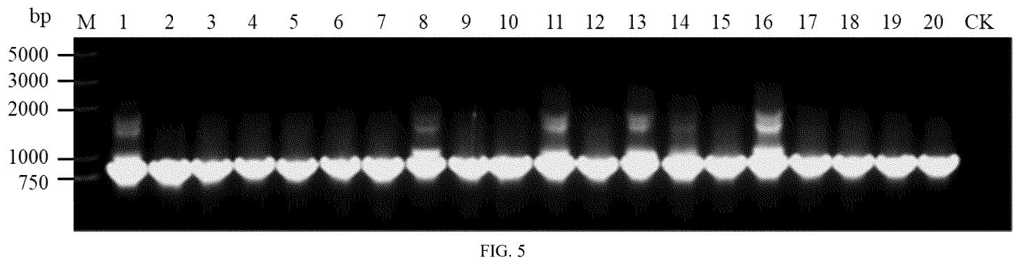

1) The vector pHENHi (Peschen D, Li H P, et al. Fusion proteins comprising a Fusarium-specific antibody linked to antifungal peptides protect plants against a fungal pathogen. Nat. Biotechnol. 2004, 22:732-738, as shown in FIG. 4) and the scFv fragment obtained in Example 3 were respectively double-digested with SfiI and NotI restriction endonucleases (purchased from NEB, and operated according to the instructions of the enzymes).
2) The pHENHi vector and scFv fragment after enzyme digestion in step 1) were purified by a DNA gel recovery kit (purchased from TIANGEN, and operated according to the instructions of the kit).
3) T4 DNA ligase (purchased from NEB, and operated according to the instructions of the enzyme) was used to connect the pHENHi vector and scFv fragment recovered in step 2) to obtain the enzyme-ligated product pHENHi-scFv.

### Example 5: Construction of Single-Chain Antibody Gene Library

1) Electroporation of enzyme-ligated products

The competent E. coli XL1-Blue MRF′ cells preserved at −80° C. were taken out and thawed on ice. Then, 5 μL of the enzyme-ligated product pHENHi-scFv in Example 4 was added to each tube of competent cells (100 μL). After careful and slight mixing, the cells were placed on ice for 3 min. Then, the competent cells were transferred to an electroporation cup (0.2 cm) (purchased from BIO-RAD) pre-cooled on ice and electroporated using the MicroPulser™ electroporation instrument of BIO-RAD (Bacteria Ec2 program was set). After transformation, 1 mL of SOC medium (composition: 2% (W/V) tryptone, 0.5% (W/V) yeast extract, 0.05% (W/V) NaCl, 20 mM glucose, pH 7.0) was immediately added to the electroporation cup, and the bacteria solution was transferred to a centrifuge tube and cultured at 37° C. and 200 r/min for 1 h to allow the cells to recover.
2) Coating of electroporated competent cells for culture
100 μL of the competent cells recovered in step 1) were coated on LB solid medium plates containing 1% (W/V) glucose and 100 μg/mL ampicillin (Amp) to count the number of monoclonal colonies. The remaining bacteria solution was centrifuged at 6000 r/min for 1 min, and part of the supernatant was discarded. About 130 μL of supernatant was retained in each tube to resuspend the bacteria, coated on LB solid culture medium plates containing 1% (W/V) glucose and 100 μg/mL Amp, and placed in a 37° C. incubator for overnight culture for 12 to 16 h until monoclonal colonies grew.
3) Collection and preservation of antibody gene library

The number of monoclonal colonies grown after electroporated competent cells were coated and cultured was counted to estimate the capacity of the antibody gene library. The colonies grown on the LB solid medium plates were collected, and an equal volume of 50% (V/V) glycerol was added and preserved in a −80° C. refrigerator.

Through the above steps, the capacity of the SARS-COV-2 N protein-specific single-chain antibody gene library finally constructed was approximately 8.7×10⁵ cfu.

### Example 6: Identification of Single-Chain Antibody Gene Library

1) 20 monoclonal colonies from the transformants grown on the plates of Example 5 were randomly selected and inoculated into LB liquid medium containing 1% (W/V) glucose and 100 μg/mL Amp, and cultured overnight at 37° C. with shaking at 220 r/min for 16 h.
2) The bacteria solution obtained in step 1) was used as a template to perform PCR amplification using the forward primer pHENpeL (GCAGCCGCTGGATTG TTATTACTCGC) set forth in SEQ ID NO: 30 and the reverse primer pHENmyc (ATTCAGATCCTCTTCTGAGATGAG) set forth in SEQ ID NO: 31. The 25 μL PCR reaction system contained 2 μL bacteria solution, 12.5 μL 2×Taq mixture, 1 μL forward primer pHENpeL (10 μM), and 1 μL reverse primer pHENmyc (10 μM). The PCR reaction conditions were as follows: 95° C. for 5 min; 94° C. for 1 min, 55° C. for 30 s, and 72° C. for 1 min, for 30 cycles; and finally 72° C. for 5 min. The PCR products were subjected to 1% (W/V) agarose gel electrophoresis to detect the positive rate of the antibody gene library (as shown in FIG. 5).

The identification results show that the positive rate of the constructed antibody gene library reaches 100%, which indicates that the constructed library has higher quality and was used for screening the specific single-chain antibody.

### Example 7: Screening of Single-Chain Antibody Gene Library Using Phage Display Technology

1) 500 μL of the bacteria solution (antibody gene library) collected and preserved in Example 5 was added to 50 mL of 2×TY medium (compositions: 1.6% (W/V) tryptone, 1% (W/V) yeast extract, 0.5% (W/V) NaCl, pH 7.0) containing 1% (W/V) glucose and 100 μg/mL Amp, cultured at 37° C., and shaken at 200 r/min until OD_{600 nm} reached 0.5.
2) 5 mL of bacteria solution was taken into a 50 mL centrifuge tube, and 60 μL of VCSM13 helper phage (the amount of phage was 20 times that of E. coli) was added. After mixing, the tube was placed in a 37° C. water bath for 30 min.
3) The mixture was centrifuged at 4000 r/min for 10 min, the supernatant was discarded, and then the mixture was resuspended in 140 mL of 2×TY medium containing 100 μg/mL Amp and 25 μg/mL kanamycin (Kan), and cultured overnight at 30° C. and 200 r/min for at least 15 h.
4) The overnight cultured bacteria solution was subpackaged in 50 mL centrifuge tubes and centrifuged at 4000 r/min at 4° C. for 30 min.
5) The supernatant was collected, and ⅕ volume of PEG/NaCl solution (20% (W/V) polyethylene glycol (PEG) 8000, 2.5 M NaCl) was added. After uniform mixing, the mixture was placed on ice for precipitation for 1 h.
6) The mixture was centrifuged at 8000 r/min at 4° C. for 30 min, the supernatant was discarded, the precipitate was resuspended in 40 mL of sterile water, and ⅕ volume of PEG/NaCl solution was immediately added. After uniform mixing, the mixture was placed at 4° C. for 20 min.
7) The mixture was centrifuged at 4000 r/min at 4° C. for 30 min, and the supernatant was discarded.
8) The residual PEG/NaCl solution was removed by gentle centrifugation.
9) 1.6 mL of sterile water was added to resuspend the precipitate, and the mixture was centrifuged at 4000 r/min for 10 min at 4° C. The supernatant was filtered through a 0.22 μm filter membrane and preserved at 4° C.
10) ELISA plates (10 wells in total, 100 μL per well, the antigen concentrations in the second, third, and fourth rounds of panning were respectively reduced to 15 ng/μL, 10 ng/μL, and 5 ng/μL) were coated with 20 ng/μL recombinant SARS-COV-2 N protein and incubated in a 37° C. water bath for 2 h.
11) Phosphate buffered saline (PBS) (compositions: 137 mM NaCl, 2.7 mM KCl, 10 mM Na₂HPO₄, 1.8 mM KH₂PO₄, pH 7.2-7.4) was used for washing 3 times.
12) 180 μL of blocking solution (PBS solution containing 2% (W/V) BSA) was added to each well and blocked in a 37° C. water bath for 2 h. A blank well was also blocked with blocking solution as a negative control.
13) 200 μL PBS was used for washing 3 times, 1 min each time.
14) 100 μL of the supernatant (phage) preserved in step 9) was added to each well and incubated in a 37° C. water bath for 2 h.
15) 200 μL PBST (PBS containing 0.1% (V/V) Tween 20) and PBS were used for washing 5 times, 3 min each time (the number of washes was increased to 10 times, 15 times, and 20 times in the second, third, and fourth rounds of panning).
16) 100 μL of triethylamine solution (100 mM) was added to each well and left at room temperature for 10 min.
17) 50 μL of Tris-HCl solution (1 M, pH 7.4) was immediately added for neutralization and transferred to a 50 mL centrifuge tube.
18) 6 mL of E. coli XL1-Blue MRF′ grown to an OD_{600 nm} of 0.7 at 37° C. and 230 r/min shaking culture conditions was added to a 50 mL centrifuge tube and infected in a 37° C. water bath for 30 min.
19) The mixture was centrifuged at 4000 r/min for 10 min, and the supernatant was discarded.
20) The bacteria was resuspended by adding 800 μL of 2×TY medium, and then coated on TYE solid medium (components: 1% (W/V) tryptone, 0.5% (W/V) yeast extract, 0.8% (W/V) NaCl, 1.5% (W/V) agar, pH 7.0) plates and cultured in a 37° C. incubator overnight until colonies grew.
21) The colonies grown on the TYE solid medium plates were collected for the next round of panning or added with an equal volume of 50% (V/V) glycerol and preserved in a −80° C. refrigerator.

### Example 8: Identification of Panning Antibody Library by Phage ELISA

1) 180 μL of 2×TY medium containing 1% (W/V) glucose and 100 μg/mL Amp was added to the wells of a 96-well cell culture plate, and 96 monoclonal clones were randomly picked from the colonies of the third round of panning antibody library with a sterile toothpick for inoculation. The clones were cultured overnight at 30° C. and 150 r/min for 16 h.
2) 180 μL of 2×TY medium (containing 1% (W/V) glucose, 100 μg/mL Amp) was added to the wells of another 96-well culture plate, 20 μL of overnight cultured bacteria solution was added, followed by shaking culture at 37° C. and 150 r/min for 4 h.
3) The mixture was centrifuged at 1800 r/min for 10 min at room temperature, and the supernatant was discarded.
4) 180 μL of 2×TY medium containing 1% (W/V) glucose, 100 μg/mL Amp, and 1 mM IPTG was added to the culture plate wells and cultured at 30° C. and 150 r/min for 14 h.
5) The mixture was centrifuged at 1800 r/min for 10 min, and the medium supernatant was used for phage ELISA detection.
6) 100 μL of recombinant SARS-COV-2 N protein (5 μg/mL) or PBS (control) was added to the ELISA plate wells and coated in a 37° C. water bath for 2 h.
7) The mixture was washed 3 times with 200 μL PBS.
8) 200 μL of blocking solution (PBS solution containing 2% (W/V) BSA) was added and blocked in a 37° C. water bath for 2 h.
9) The mixture was washed 3 times with 200 μL PBS.
10) 100 μL of the medium supernatant collected in the step 5 was added and reacted at 37° C. for 2 h.
11) The mixture was washed 3 times with PBST and PBS.
12) 100 μL of 1:5000 (V/V) diluted anti-c-myc mouse monoclonal antibody was added and reacted at 37° C. for 1.5 h.
13) The mixture was washed 3 times with 200 μL PBST and PBS.
14) 100 μL of 1:5000 (V/V) diluted AP-labeled goat anti-mouse antibody was added and reacted at 37° C. for 1 h.
15) The mixture was washed 5 times with 200 μL PBST and PBS.
16) 100 μL of 0.2% (W/V) pNPP color developing solution was added and reacted in the dark for 15-30 min.
17) 50 μL NaOH solution (3 M) was added to terminate the reaction, and the OD_{405 nm} reading was measured using a microplate reader.

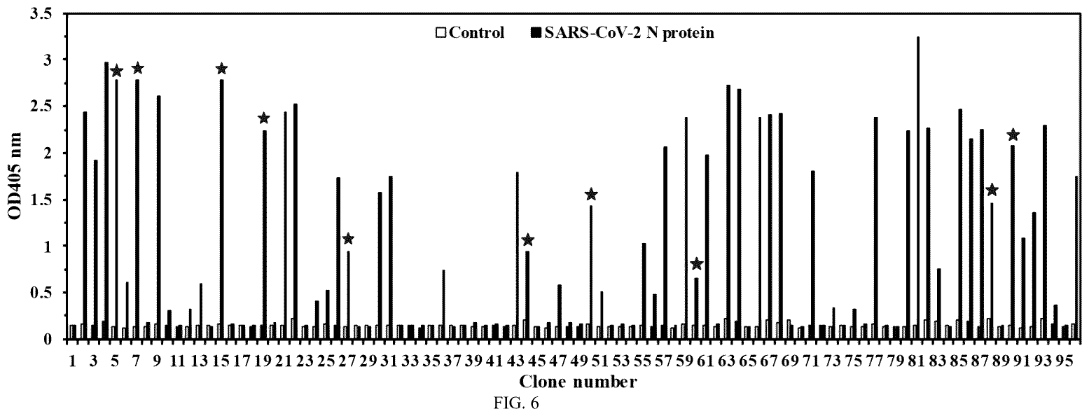

The results of phase ELISA identification show that 57 of 96 monoclonal samples develop color (as shown in FIG. 6), and cultured bacteria solution of 10 monoclonal samples (marked with * in FIG. 6) with higher color development (the degree of color development is proportional to OD_{405 nm} by the microplate reader, and the color development is quantified by OD_{405 nm} by the microplate reader) are picked up and sent to Sangon Biotech (Shanghai) Co., Ltd. for sequencing. The analysis results show that the sequences of the strains are different. According to the phage ELISA results and sequence analysis, the gene consists of 753 nucleotides, and the selected nucleotide sequence is set forth in SEQ ID NO: 1 in a sequence listing.

ATGGCCGAGG TGAAGCTGGC GGAATCTGGG CCTGAGCTGG TGAGGCCTGG GGTCTCAGTG 60
AAGATTTCCT GCAAGGGTTC CGGCTACACA TTCACTGATT ATCCTATGCA CTGGGTGAAG 120
CAGAGTCATG CAAAGAGTCT AGAGTGGATT GGAGTTATTA GTACTTACTC TGGTAATACA 180
AACTACAACC AGAAGTTTAG GGGCAAGGCC ACAATGACTG TAGACAAATC CTCCAGCACA 240
GCCTATATGG AACTTGCCAG ATTGACATCT GAGGATTCTG CCATCTATTA CTGTACAAGA 300
GGGGGTAACT ACGGGTTTGA CCCCTGGGGC CAAGGCACCA CTCTCACAGT CTCCTCAGCC 360
AAAACAACAG CCCCATCTGT CGGTGGCGGC GGTAGCGGCG GTGGCGGTTC TGGAGGCGGC 420
GGTTCTGACA TTCAGATGAC ACAGTCTCAC AAATTCTTGT CCACATCAGT AGGAGACAGG 480
GTCAACATCA CCTGCAAGGC CAGTCAGGAT GTGGGTACTG CTGTAGCCTG GTATCAACAG 540
AAACCAGGGC AATCTCCTAA ACTGCTGATT TACTGGGCAT CCACCCGGCA CACTGGAGTC 600
CCTGATCGCT TCACAGGCAG TGGATCTGGG ACAGATTTCA CTCTCACCAT TACCAATGTG 660
CAGTCTGAAG ACTTGGCAGA ATATTTCTGT CAGCAATATA GCAGCTATCC TCTCACGTTC 720
GGCTCGGGGA CAAAGATAGA AATGAAACGT GCT 753

The heavy chain variable region V_{H} consists of 90 nucleotides (comprising three variable regions CDR H1, CDR H2, CDR H3) and has a sequence set forth in SEQ ID NO: 3:

GATTATCCTA TGCAC
CDR H1
GTTATTAGTA CTTACTCTGG TAATACAAAC
TACAACCAGA AGTTTAGGGG C
CDR H2
GGGGGTAACT ACGGGTTTGA CCCC
CDR H3

The light chain variable region V_{L} consists of 81 nucleotides (comprising three variable regions CDR L1, CDR L2, CDR L3) and has a sequence set forth in SEQ ID NO: 5:

AAGGCCAGTC AGGATGTGGG TACTGCTGTA GCC
CDR L1
TGGGCATCCA CCCGGCACAC T
CDR L2
CAGCAATATA GCAGCTATCC TCTCACG
CDR L3

The amino acid sequence of the gene is set forth in SEQ ID NO: 2 in a sequence listing and consists of 251 amino acids.

SEQ ID NO: 2
MAEVKLAESGPELVRPGVSVKISCKGSGYTFTDYPMHWVKQSHAKS
LEWIGVISTYSGNTNYNQKFRGKATMTVDKSSSTAYMELARLTSE
DSAIYYCTRGGNYGFDPWGQGTTLTVSSAKTTAPSVGGGGSGGGG
SGGGGSDIQMTQSHKFLSTSVGDRVNITCKASQDVGTAVAWYQQK
PGQSPKLLIYWASTRHTGVPDRFTGSGSGTDFTLTITNVQSEDLA
EYFCQQYSSYPLTFGSGTKIEMKRA

The gene mainly consists of a heavy chain variable region V_{H}, a light chain variable region V_{L} and a linker, wherein the V_{H} and the V_{L} are connected through the linker (Gly₄Ser)₃ to jointly complete the recognition and the binding of SARS-COV-2 N protein. The heavy chain variable region V_{H} of the gene consists of 30 amino acids (comprising three variable regions CDR H1, CDR H2, CDR H3), and has a sequence set forth in SEQ ID NO: 4:

DYPMH
CDR H1
VISTYSGNTNYNQKFRG
CDR H2
GGN
YGFDP
CDR H3

The light chain variable region V_{L} consists of 27 amino acids (comprising three variable regions CDR L1, CDR L2, CDR L3), and has a sequence set forth in SEQ ID NO: 6:

KASQDVGTAVA
CDRL1
WASTRHT
CDRL2
QQYSSYPLT
CDRL3

The E. coli containing the gene was named recombinant E. coli XL1-Blue MRF′/pHENHi-H6, and the monoclonal cultured bacteria solution was added with an equal volume of 50% (V/V) glycerol and preserved in a −80° C. refrigerator.

### Example 9: Large-Scale Expression and Purification of H6 Antibody by Recombinant E. coli

1) 5 μL of the recombinant E. coli XL1-Blue MRF′/pHENHi-H6 preserved in glycerol in Example 8 was inoculated into 20 mL of 2×TY medium containing 100 μg/mL Amp, and cultured overnight at 37° C. and 200 r/min for 12 h.
2) 2 mL of overnight cultured bacteria solution was added to 200 mL of 2×TY medium containing 100 μg/mL Amp, and the mixture was shaken and cultured at 37° C. and 200 r/min until OD_{600 nm} reached 0.5.
3) IPTG was added at a final concentration of 1 mM, and the mixture was induced at 30° C. and 200 r/min for expression for 16 h.
4) The cultured bacteria solution was subpackaged into 50 mL centrifuge tubes and centrifuged at 8000 r/min for 10 min at 4° C., and the supernatant was discarded.
5) 10 mL of buffer A (50 mM NaH₂PO₄, 300 mM NaCl, pH 8.0) was added, resuspended, sonicated (35%, sonicated for 3 s, stopped for 3 s, repeated 400 times), and centrifuged at 12000 r/min for 15 min; periplasmic proteins were extracted, and the supernatant was filtered with a 0.22 μm filter and preserved at 4° C. for later use.
6) The purification column (purchased from BIO-RAD) was mounted, and 400 μL of well-mixed matrix (purchased from QIAGEN) was added and allowed to stand for more than 30 min.
7) The lower seal was cut off, the liquid was allowed to flow down, and 5 mL of buffer A was used for equilibration.
8) The single-chain antibody sample obtained in the step 5) was added for column chromatography, and the effluent of the sample after the column chromatography was collected and preserved.
9) 1 mL of buffer B (50 mM NaH₂PO₄, 300 mM NaCl, 20 mM imidazole, pH 8.0) was added to elute the purification column 3 times, and the eluates were collected as B1, B2, and B3 (impure proteins), respectively.
10) 500 μL of buffer C (50 mM NaH₂PO₄, 300 mM NaCl, 250 mM imidazole, pH 8.0) was added to elute the purification column 3 times, and the eluates were collected as C1, C2, and C3 (H6 antibody), respectively.
11) After 5 mL of buffer A was added to equilibrate the purification column, 1 mL of 30% (V/V) alcohol was added, and the purification column was preserved at 4° C.
12) The recombinant antibody was detected by SDS-PAGE electrophoresis (Example 10) and then dialyzed against PBS.

### Example 10: Detection of Purified H6 Antibody by SDS-PAGE Electrophoresis

1) 30 μL of the purified H6 antibody in Example 9 was taken, and 7.5 μL of 5× sodium dodecyl sulfate (SDS) loading buffer (250 mM Tris-HCl (pH 6.8), 10% (W/V) SDS (electrophoresis grade), 0.5% (W/V) bromophenol blue, 50% (V/V) glycerol, 5% (W/V) β-mercaptoethanol) was added, mixed well, boiled in a water bath for 8 min, and then placed on ice for later use.
2) The separation gel and the stacking gel were prepared according to the SDS-PAGE gel preparation kit (purchased from Solarbio Science & Technology Co., Ltd.).
3) A protein vertical electrophoresis system (purchased from BIO-RAD) was mounted, 1× Tris-glycine electrophoresis buffer (25 mM Tris, 250 mM glycine, 0.1% (W/V) SDS) was added, and the sample placed on ice was loaded.
4) The electrophoresis was performed at 80 V for 30 min, and then the electrophoresis was performed at 120 V for 100 min until bromophenol blue runs out of the separation gel.
5) The gel was removed, and the stacking gel was removed and stained with staining solution (0.1% (W/V) Coomassie Brilliant Blue R-250, 25% (V/V) isopropanol, 10% (V/V) glacial acetic acid) for 30 min.
6) Destaining was performed 4 times with a destaining solution (5% (V/V) methanol, 7.5% (V/V) glacial acetic acid), and observation and photography were performed.

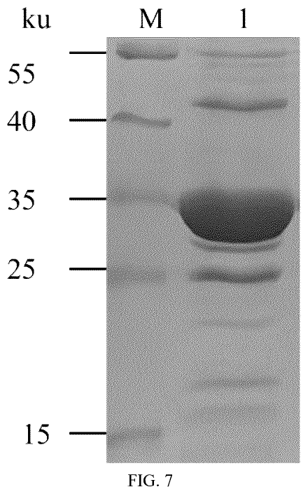

The results of SDS-PAGE electrophoresis are shown in FIG. 7, where a protein of about 35 ku in size can be seen.

### Example 11: Analysis of the Binding Properties of H6 Antibody to Recombinant SARS-COV-2 N Protein by Surface Plasmon Resonance (SPR)

The purified protein of Example 9 and recombinant SARS-COV-2 N protein were sent to UA Biotech R&D Co., Ltd. (Nanjing, China) for SPR analysis of the binding affinity of H6 to recombinant SARS-COV-2 N protein.

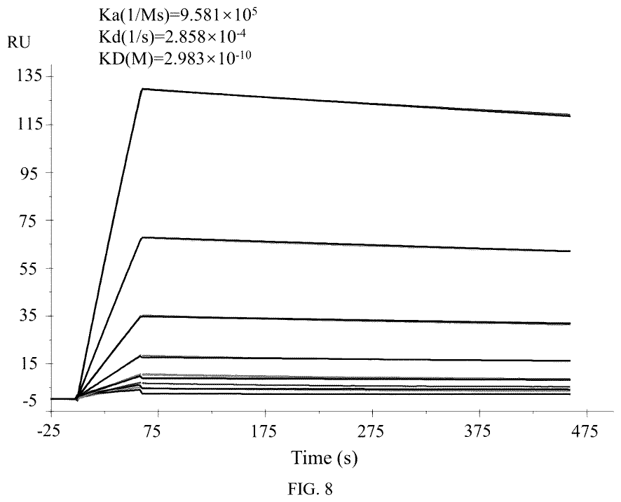

The SPR analysis results are shown in FIG. 8, and the measured kinetic parameters are shown in Table 5.

TABLE 5
Kinetic parameters

| Ka (1/Ms) | Kd (1/s) | KD (M) |
|---|---|---|
| 9.581 × 10⁵ | 2.858 × 10⁻⁴ | 2.983 × 10⁻¹⁰ |

Note:
Ka, binding constant;
Kd, dissociation constant;
KD, kinetic constant (KD = kd/ka)

The SPR analysis results show that the SARS-COV-2 N protein-specific single-chain antibody H6 has a strong binding ability to the N protein.

### Example 12: Construction, Prokaryotic Expression and Purification of H6-AP Fusion Protein Expression Vector

1) The H6 antibody gene was amplified by SOE-PCR with 218 linker added to the 3′ end (Whitlow M, Bell B A, et al. An improved Linker for single-chain Fv with reduced aggregation and enhanced proteolytic stability. Protein. Eng. 1993, 6:989-995.), and digested with SfiI and NotI endonucleases (purchased from NEB, and operated according to the instructions of the enzymes) and directly ligated to the pDAP2/S vector (Kerschbaumer R J, Hirschl S, et al. Single-chain Fv fusion proteins suitable as coating and detecting reagents in a double antibody sandwich Enzyme-linked immunosorbent assay. Anal. Biochem., 1997, 249:219-227.) to obtain a recombinant plasmid containing the H6-AP fusion protein gene, which was electroporated into competent E. coli XL1-Blue MRF′ cells (see the step 1 in Example 5). After the single clone was selected and cultured, the single clone was sent to Sangon Biotech (Shanghai) Co., Ltd. for sequencing and identification of the positive clones to obtain recombinant E. coli containing recombinant plasmid DNA.
2) 5 μL of the recombinant E. coli containing recombinant plasmid DNA in step 1) was inoculated into 20 mL of 2×TY medium containing 100 μg/mL Amp, and cultured overnight at 37° C. with shaking at 200 r/min for 12 h.
3) The plasmid was extracted using the Omega plasmid extraction kit (purchased from Omega Biotechnology, USA) and electroporated into competent BL21 (DE3) cells (see step 1 in Example 5 for the method).
4) Expression and purification of H6-AP fusion protein were performed according to the method of Example 9, except that in step 3), the final concentration of IPTG was 0.5 mM, the temperature was 24° C., and the induction expression was 3 h.

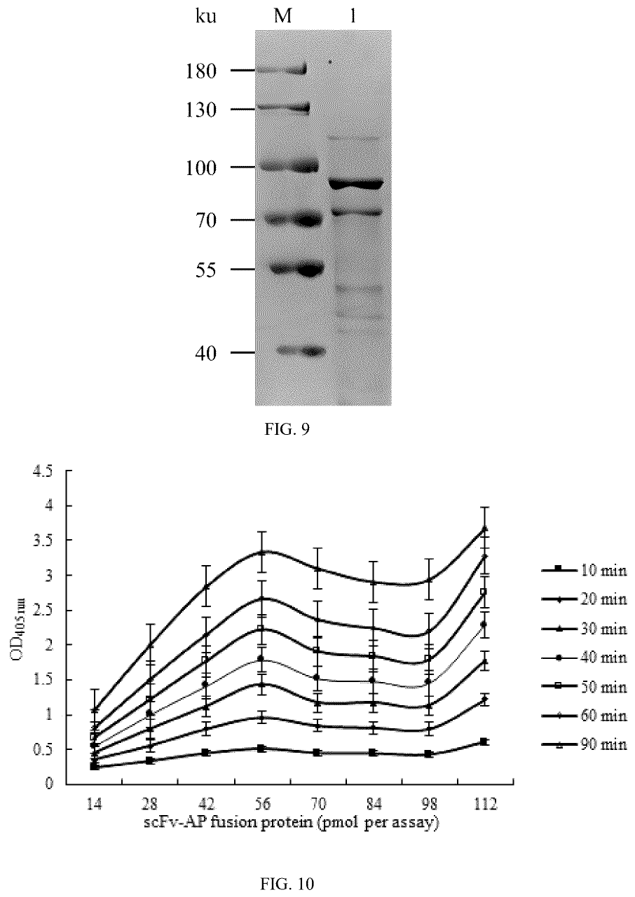

After the H6-AP fusion protein was expressed and purified, the results of SDS-PAGE electrophoresis were shown in FIG. 9, which shows a target protein of about 90 ku in size, whose amino acid sequence is set forth in SEQ ID NO: 8, and whose nucleotide sequence is set forth in SEQ ID NO: 7.

SEQ ID NO: 7
ATGGCAGAGG TAAAACTAGC TGAATCAGGA CCAGAGTTGG TTCGTCCGGG TGTGAGCGTG 60
AAGATCAGCT GTAAAGGTTC TGGCTATACC TTTACCGACT ACCCGATGCA TTGGGTTAAG 120
CAGAGCCACG CAAAGAGCCT GGAATGGATT GGTGTCATCA GCACCTACAG CGGCAACACC 180
AACTACAACC AGAAGTTCCG CGGTAAGGCT ACCATGACCG TGGACAAAAG CTCATCTACT 240
GCCTATATGG AACTTGCGCG TCTGACCTCC GAGGACAGCG CGATTTACTA CTGCACCAGA 300
GGTGGTAACT ATGGTTTTGA TCCGTGGGGT CAAGGTACTA CGTTGACGGT GTCTAGCGCG 360
AAGACCACCG CTCCGAGCGT TGGTGGCGGT GGTTCGGGCG GTGGTGGCTC GGGCGGCGGT 420
GGCTCTGATA TTCAGATGAC CCAGTCCCAT AAATTTCTGA GCACCAGCGT CGGTGATCGT 480
GTAAATATCA CGTGCAAGGC GAGCCAAGAT GTGGGTACGG CCGTTGCATG GTATCAGCAA 540
AAACCGGGCC AATCCCCGAA ACTGCTGATC TACTGGGCAA GCACTCGCCA CACCGGTGTT 600
CCGGATCGTT TTACCGGCTC CGGCTCCGGC ACTGACTTCA CCTTGACCAT TACCAATGTT 660
CAGAGCGAGG ACCTGGCGGA ATACTTCTGC CAGCAATATT CTAGCTATCC GCTGACCTTC 720
GGCTCGGGCA CGAAAATCGA GATGAAACGT GCGGTGGACA GCTCCGGTAG CACCAGCGGC 780
TCCGGCAAGC CGGGTTCCGG CGAAGGTTCT ACGGCGGCCG CAGCCCGGGC ACCAGAAATG 840
CCTGTTCTGG AAAACCGGGC TGCTCAGGGC GATATTACTG CACCCGGCGG TGCTCGCCGT 900
TTAACGGGTG ATCAGACTGC CGCTCTGCGT GATTCTCTTA GCGATAAACC TGCAAAAAAT 960
ATTATTTTGC TGATTGGCGA TGGGATGGGG GACTCGGAAA TTACTGCCGC ACGTAATTAT 1020
GCCGAAGGTG CGGGCGGCTT TTTTAAAGGT ATAGATGCCT TACCGCTTAC CGGGCAATAC 1080
ACTCACTATG CGCTGAATAA AAAAACCGGC AAACCGGACT ACGTCACCTC CTCGGCTGCA 1140
TCAGCAACCG CCTGGTCAAC CGGTGTCAAA ACCTATAACG GCGCGCTGGG CGTCGATATT 1200
CACGAAAAAG ATCACCCAAC GATTCTGGAA ATGGCAAAAG CCGCAGGTCT GGCGACCGGT 1260
AACGTTTCTA CCGCAGAGTT GCAGGATGCC ACGCCCGCTG CGCTGGTGGC ACATGTGACC 1320
TCGCGCAAAT GCTACGGTCC GAGCGCGACC AGTGAAAAAT GTCCGGGTAA CGCTCTGGAA 1380
AAAGGCGGAA AAGGATCGAT TACCGAACAG CTGCTTAACG CTCGTGCCGA CGTTACGCTT 1440
GGCGGCGGCG CAAAAACCTT TGCTGAAACG GCAACCGCTG GTGAATGGCA GGGAAAAACG 1500
CTGCGTGAAC AGGCACAGGC GCGTGGTTAT CAGTTGGTGA GCGATGCTGC CTCACTGAAT 1560
TCGGTGACGG AAGCGAATCA GCAAAAACCC CTGCTTGGCC TGTTTGCTGA CGGCAATATG 1620
CCAGTGCGCT GGCTAGGACC GAAAGCAACG TACCATGGCA ATATCGATAA GCCCGCAGTC 1680
ACCTGTACGC CAAATCCGCA ACGTAATGAC AGTGTACCAA CCCTGGCGCA GATGACCGAC 1740
AAAGCCATTG AATTGTTGAG TAAAAATGAG AAAGGCTTTT TCCTGCAAGT TGAAGGTGCG 1800
TCAATCGATA AACAGGATCA TGCTGCGAAT CCTTGTGGGC AAATTGGCGA GACGGTCGAT 1860
CTCGATGAAG CCGTACAACG GGCGCTGGAA TTCGCTAAAAAGGAGGGTAA CACGCTGGTC 1920
ATAGTCACCG CTGATCACGC CCACGCCAGC CAGATTGTTG CGCCGGATAC CAAAGCTCCG 1980
GGCCTCACCC AGGCGCTAAA TACCAAAGAT GGCGCAGTGA TGGTGATGAG TTACGGGAAC 2040
TCCGAAGAGG ATTCACAAGA ACATACCGGC AGTCAGTTGC GTATTGCGGC GTATGGCCCG 2100
CATGCCGCCA ATGTTGTTGG ACTGACCGAC CAGACCGATC TCTTCTACAC CATGAAAGCC 2160
GCTCTGGGGG ATATCGCACA CCATCACCAT CACCAT 2196
SEQ ID NO: 8
MAEVKLAESGPELVRPGVSVKISCKGSGYTFTDYPMHWVKQSHAKSLEWIGVISTYSGNTNYNQKFRGKA
TMTVDKSSSTAYMELARLTSEDSAIYYCTRGGNYGFDPWGQGTTLTVSSAKTTAPSVGGGGSGGGGSGGG
GSDIQMTQSHKFLSTSVGDRVNITCKASQDVGTAVAWYQQKPGQSPKLLIYWASTRHTGVPDRFTGSGSG
TDFTLTITNVQSEDLAEYFCQQYSSYPLTFGSGTKIEMKRAVDSSGSTSGSGKPGSGEGSTAAAARAPEM
PVLENRAAQGDITAPGGARRLTGDQTAALRDSLSDKPAKNIILLIGDGMGDSEITAARNYAEGAGGFFKG
IDALPLTGQYTHYALNKKTGKPDYVTSSAASATAWSTGVKTYNGALGVDIHEKDHPTILEMAKAAGLATG
NVSTAELQDATPAALVAHVTSRKCYGPSATSEKCPGNALEKGGKGSITEQLLNARADVTLGGGAKTFAET
ATAGEWQGKTLREQAQARGYQLVSDAASLNSVTEANQQKPLLGLFADGNMPVRWLGPKATYHGNIDKPAV
TCTPNPQRNDSVPTLAQMTDKAIELLSKNEKGFFLQVEGASIDKQDHAANPCGQIGETVDLDEAVQRALE
FAKKEGNTLVIVTADHAHASQIVAPDTKAPGLTQALNTKDGAVMVMSYGNSEEDSQEHTGSQLRIAAYGP
HAANVVGLTDQTDLFYTMKAALGDIAHHHHHH

### Example 13: Activity Detection of H6-AP Fusion Protein

1) 100 μL of antigen recombinant SARS-COV-2 N protein (5 μg/mL) was added to the ELISA plate wells and incubated at 37° C. for 2 h.
2) The protein was washed 3 times with 200 μL PBS.
3) 200 μL of blocking solution was added and blocked at 37° C. for 2 h, and blank wells were blocked as controls.
4) The mixture was washed 3 times with 200 μL PBS.
5) 100 μL of the H6-AP fusion protein purified in Example 12 and diluted in 100 μL of blocking solution was added to each well and incubated at 37° C. for 2 h.
6) The mixture was washed 3 times with 200 μL PBST and PBS.
7) 100 μL of 0.2% (W/V) pNPP color developing solution was added and the color was developed for 30 min, and the OD_{405 nm} value was read.

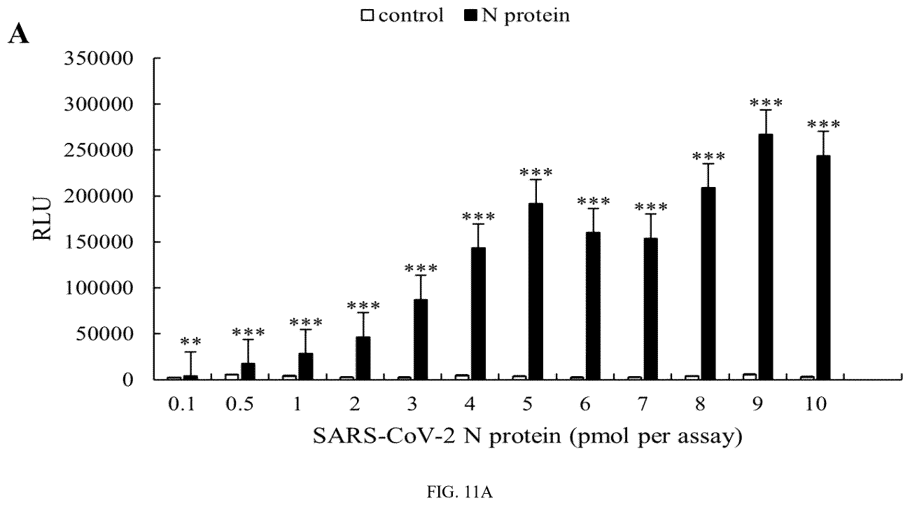

ELISA results show that the H6-AP fusion protein still maintains a strong binding ability to the recombinant SARS-COV-2 N protein, the average OD_{405 nm} reading value (1.773) reaches 16 times that of the control group (0.109) when the significant difference is the largest, the color development intensity of an antigen-containing group is gradually increased along with the increase of reaction time, the control group has no obvious change, and the maximum significance difference is increased from 16 times to 31 times at 90 min (FIG. 10).

### Example 14: Detection Limit of H6-AP Fusion Protein for Detecting SARS-COV-2 N Protein

1) 100 μL of antigen recombinant SARS-COV-2 N protein (1 μmol, 2 μmol, 3 μmol, 4 μmol, 5 μmol, 6 μmol, 7 μmol, 8 μmol, 9 μmol, 10 μmol) was added to the ELISA plate wells and incubated at 37° C. for 2 h.
2) The protein was washed 3 times with 200 μL PBS.
3) 200 μL of blocking solution was added and blocked at 37° C. for 2 h, and blank wells were blocked as controls.
4) The mixture was washed 3 times with 200 μL PBS.
5) The H6-AP fusion protein (70 μmol) purified in Example 12 and diluted in 100 μL of blocking solution was added to each well and incubated at 37° C. for 2 h.
6) The mixture was washed 3 times with 200 μL PBST and PBS.
7) 100 μL of 0.2% (W/V) pNPP color developing solution was added and the color was developed for 20 min, and the OD_{405 nm} value was read.

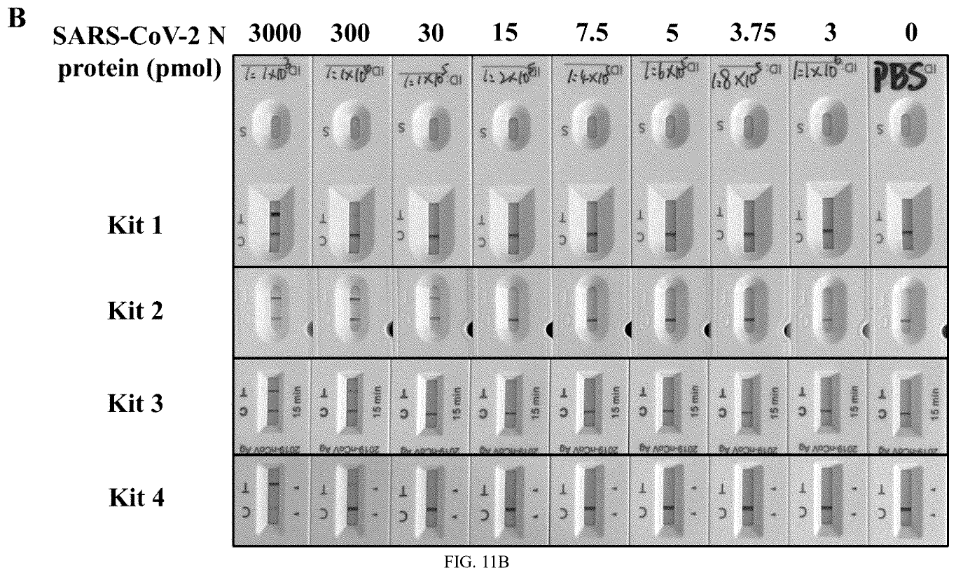

The ELISA results show (as shown in FIGS. 11A-11B) that the detection limit of the H6-AP fusion protein for the recombinant SARS-COV-2 N protein decreases with the decrease of the molecular weight of SARS-COV-2 N protein, and the OD_{405 nm} value decreases, and the lowest detection limit can be as low as 3 pmol (the detection result at 2 pmol is not statistically significant, and thus the lowest detection limit is defined as 3 μmol). The present invention is further compared with commercially available rapid detection kits of 4 different brands (Kit 1: Cofoe Medical Technology Co., Ltd., Kit 2: Xiamen AmonMed Biotechnology Co., Ltd., Kit 3: Winner Medical Co., Ltd., Kit 4: Guangzhou Wondfo Biotech Co., Ltd.), the recombinant SARS-COV-2 N protein is subjected to gradient dilution, and the detection limit of the rapid detection kit is only 30 pmol at the lowest, which is higher than the detection limit of 3 pmol of the present invention.

### Example 15: Minimum Dosage of H6-AP Fusion Protein for Detecting SARS-COV-2 N Protein

1) 100 μL of antigen recombinant SARS-COV-2 N protein (10 μmol) was added to the ELISA plate wells and incubated at 37° C. for 2 h.
2) The protein was washed 3 times with 200 μL PBS.
3) 200 μL of blocking solution was added and blocked at 37° C. for 2 h, and blank wells were blocked as controls.
4) The mixture was washed 3 times with 200 μL PBS.
5) The H6-AP fusion protein (0.68 μmol, 4.26 μmol, 6.8 μmol, 14 μmol, 28 μmol, 42 μmol, 56 μmol, 70 μmol, 84 μmol, 98 μmol, 112 μmol) purified in Example 12 and diluted in 100 μL of blocking solution was added to each well and incubated at 37° C. for 2 h.
6) The mixture was washed 3 times with 200 μL PBST and PBS.
7) 100 μL of 0.2% (W/V) pNPP color developing solution was added and the color was developed for 20 min, and the OD_{405 nm} value was read.

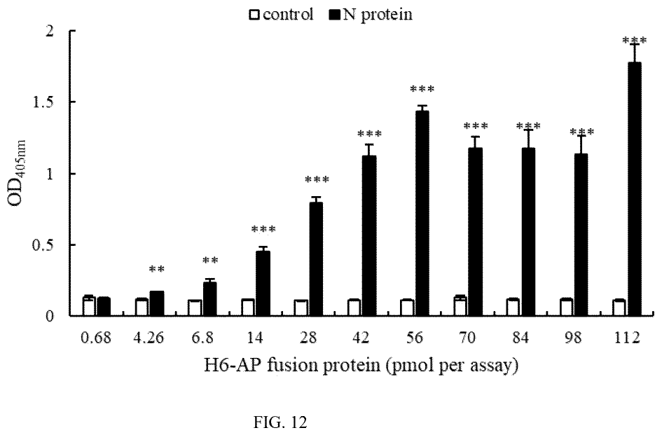

The ELISA results show (as shown in FIG. 12) that the amount of the H6-AP fusion protein for detecting the recombinant SARS-COV-2 N protein decreases with the decrease of the amount of the fusion protein, the OD_{405 nm} value decreases gradually, and the lowest amount can be as low as 4.26 pmol.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A SARS-COV-2 N protein-specific single-chain antibody, wherein the SARS-COV-2 N protein-specific single-chain antibody comprises a heavy chain variable region V_{H}, a linker, and a light chain variable region V_{L}, wherein the heavy chain variable region V_{H}, the linker, and the light chain variable region V_{L} are connected in sequence; the amino acid sequence of the heavy chain variable region V_{H} is set forth in SEQ ID NO: 4; and the amino acid sequence of the light chain variable region V_{L} is set forth in SEQ ID NO: 6.

2. The SARS-COV-2 N protein-specific single-chain antibody according to claim 1, wherein the linker is (Gly₄Ser)₃.

3. The SARS-COV-2 N protein-specific single-chain antibody according to claim 2, wherein the amino acid sequence of the SARS-COV-2 N protein-specific single-chain antibody is set forth in SEQ ID NO: 2.

4. A fusion protein for detecting a SARS-COV-2, comprising the SARS-COV-2 N protein-specific single-chain antibody according to claim 1 and an alkaline phosphatase.

5. The fusion protein for detecting the SARS-COV-2 according to claim 4, wherein the amino acid sequence of the fusion protein is set forth in SEQ ID NO: 8.

6. A preparation method for a novel coronavirus detection kit, comprising using the fusion protein according to claim 4.

7. The preparation method according to claim 6, wherein the novel coronavirus detection kit comprises the fusion protein and an auxiliary detection reagent.

8. The preparation method according to claim 7, wherein the auxiliary detection reagent comprises: a transparent ELISA plate, a washing buffer, a blocking solution, a color developing solution, and a positive control substance.

9. A nucleic acid encoding the fusion protein according to claim 4.

10. A host cell expressing the fusion protein according to claim 4 or comprising a nucleic acid encoding the fusion protein.

11. The fusion protein for detecting the SARS-COV-2 according to claim 4,
wherein in the SARS-COV-2 N protein-specific single-chain antibody, the linker is (Gly₄Ser)₃.

12. The fusion protein for detecting the SARS-COV-2 according to claim 11, wherein the amino acid sequence of the SARS-COV-2 N protein-specific single-chain antibody is set forth in SEQ ID NO: 2.

13. The preparation method according to claim 6, wherein the amino acid sequence of the fusion protein is set forth in SEQ ID NO: 8.

14. The preparation method according to claim 13, wherein the novel coronavirus detection kit comprises the fusion protein and an auxiliary detection reagent.

15. The nucleic acid according to claim 9, wherein the amino acid sequence of the fusion protein is set forth in SEQ ID NO: 8.

16. The host cell according to claim 10, wherein the amino acid sequence of the fusion protein is set forth in SEQ ID NO: 8.


SEQUENCE LISTING (31 sequences)

SEQ ID NO: 1 - DNA, 753 bp, synthetic construct
  source 1..753: mol_type = other DNA
atggccgaggtgaagctggcggaatctgggcctgagctggtgaggcctggggtctcagtgaagatttcctgcaagggttccggctacacattcactgattatcctatgcactgggtgaagcagagtcatgcaaagagtctagagtggattggagttattagtacttactctggtaatacaaactacaaccagaagtttaggggcaaggccacaatgactgtagacaaatcctccagcacagcctatatggaacttgccagattgacatctgaggattctgccatctattactgtacaagagggggtaactacgggtttgacccctggggccaaggcaccactctcacagtctcctcagccaaaacaacagccccatctgtcggtggcggcggtagcggcggtggcggttctggaggcggcggttctgacattcagatgacacagtctcacaaattcttgtccacatcagtaggagacagggtcaacatcacctgcaaggccagtcaggatgtgggtactgctgtagcctggtatcaacagaaaccagggcaatctcctaaactgctgatttactgggcatccacccggcacactggagtccctgatcgcttcacaggcagtggatctgggacagatttcactctcaccattaccaatgtgcagtctgaagacttggcagaatatttctgtcagcaatatagcagctatcctctcacgttcggctcggggacaaagatagaaatgaaacgtgct

SEQ ID NO: 2 - protein, 251 aa, synthetic construct
MAEVKLAESGPELVRPGVSVKISCKGSGYTFTDYPMHWVKQSHAKSLEWIGVISTYSGNTNYNQKFRGKATMTVDKSSSTAYMELARLTSEDSAIYYCTRGGNYGFDPWGQGTTLTVSSAKTTAPSVGGGGSGGGGSGGGGSDIQMTQSHKFLSTSVGDRVNITCKASQDVGTAVAWYQQKPGQSPKLLIYWASTRHTGVPDRFTGSGSGTDFTLTITNVQSEDLAEYFCQQYSSYPLTFGSGTKIEMKRA

SEQ ID NO: 3 - DNA, 90 bp, synthetic construct
  source 1..90: mol_type = other DNA
gattatcctatgcacgttattagtacttactctggtaatacaaactacaaccagaagtttaggggcgggggtaactacgggtttgacccc

SEQ ID NO: 4 - protein, 30 aa, synthetic construct
DYPMHVISTYSGNTNYNQKFRGGGNYGFDP

SEQ ID NO: 5 - DNA, 81 bp, synthetic construct
  source 1..81: mol_type = other DNA
aaggccagtcaggatgtgggtactgctgtagcctgggcatccacccggcacactcagcaatatagcagctatcctctcacg

SEQ ID NO: 6 - protein, 27 aa, synthetic construct
KASQDVGTAVAWASTRHTQQYSSYPLT

SEQ ID NO: 7 - DNA, 2196 bp, synthetic construct
  source 1..2196: mol_type = other DNA
atggcagaggtaaaactagctgaatcaggaccagagttggttcgtccgggtgtgagcgtgaagatcagctgtaaaggttctggctatacctttaccgactacccgatgcattgggttaagcagagccacgcaaagagcctggaatggattggtgtcatcagcacctacagcggcaacaccaactacaaccagaagttccgcggtaaggctaccatgaccgtggacaaaagctcatctactgcctatatggaacttgcgcgtctgacctccgaggacagcgcgatttactactgcaccagaggtggtaactatggttttgatccgtggggtcaaggtactacgttgacggtgtctagcgcgaagaccaccgctccgagcgttggtggcggtggttcgggcggtggtggctcgggcggcggtggctctgatattcagatgacccagtcccataaatttctgagcaccagcgtcggtgatcgtgtaaatatcacgtgcaaggcgagccaagatgtgggtacggccgttgcatggtatcagcaaaaaccgggccaatccccgaaactgctgatctactgggcaagcactcgccacaccggtgttccggatcgttttaccggctccggctccggcactgacttcaccttgaccattaccaatgttcagagcgaggacctggcggaatacttctgccagcaatattctagctatccgctgaccttcggctcgggcacgaaaatcgagatgaaacgtgcggtggacagctccggtagcaccagcggctccggcaagccgggttccggcgaaggttctacggcggccgcagcccgggcaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcacctcctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctgggggatatcgcacaccatcaccatcaccat

SEQ ID NO: 8 - protein, 732 aa, synthetic construct
MAEVKLAESGPELVRPGVSVKISCKGSGYTFTDYPMHWVKQSHAKSLEWIGVISTYSGNTNYNQKFRGKATMTVDKSSSTAYMELARLTSEDSAIYYCTRGGNYGFDPWGQGTTLTVSSAKTTAPSVGGGGSGGGGSGGGGSDIQMTQSHKFLSTSVGDRVNITCKASQDVGTAVAWYQQKPGQSPKLLIYWASTRHTGVPDRFTGSGSGTDFTLTITNVQSEDLAEYFCQQYSSYPLTFGSGTKIEMKRAVDSSGSTSGSGKPGSGEGSTAAAARAPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGDIAHHHHHH

SEQ ID NO: 9 - DNA, 58 bp, synthetic construct
  source 1..58: mol_type = other DNA
catgccatgactcgcggcccagccggccatggccgakgtrcagcttcaggagtcrgga

SEQ ID NO: 10 - DNA, 58 bp, synthetic construct
  source 1..58: mol_type = other DNA
catgccatgactcgcggcccagccggccatggcccaggtgcagctgaagsagtcwggm

SEQ ID NO: 11 - DNA, 58 bp, synthetic construct
  source 1..58: mol_type = other DNA
catgccatgactcgcggcccagccggccatggccsaggtycagctgcarcagtcwggd

SEQ ID NO: 12 - DNA, 58 bp, synthetic construct
  source 1..58: mol_type = other DNA
catgccatgactcgcggcccagccggccatggccsaggtccarctgcagsaryctggr

SEQ ID NO: 13 - DNA, 58 bp, synthetic construct
  source 1..58: mol_type = other DNA
catgccatgactcgcggcccagccggccatggccgaggttcagctgcagcagtctggg

SEQ ID NO: 14 - DNA, 58 bp, synthetic construct
  source 1..58: mol_type = other DNA
catgccatgactcgcggcccagccggccatggccgargtgaagctggtggartctggr

SEQ ID NO: 15 - DNA, 58 bp, synthetic construct
  source 1..58: mol_type = other DNA
catgccatgactcgcggcccagccggccatggccgaggtgaagcttctcgagtctgga

SEQ ID NO: 16 - DNA, 57 bp, synthetic construct
  source 1..57: mol_type = other DNA
catgccatgactcgcggcccagccggccatggccgargtgaagctkgakgagwctgr

SEQ ID NO: 17 - DNA, 58 bp, synthetic construct
  source 1..58: mol_type = other DNA
catgccatgactcgcggcccagccggccatggccgavgtgmwgctkgtggagtctggk

SEQ ID NO: 18 - DNA, 58 bp, synthetic construct
  source 1..58: mol_type = other DNA
catgccatgactcgcggcccagccggccatggccsaggtycagctkcagcagtctgga

SEQ ID NO: 19 - DNA, 57 bp, synthetic construct
  source 1..57: mol_type = other DNA
tccagaaccgccaccgccgctaccgccgccaccgacagrtggggstgtygttttggc

SEQ ID NO: 20 - DNA, 54 bp, synthetic construct
  source 1..54: mol_type = other DNA
tccagaaccgccaccgccgctaccgccgccaccgacagatggggctgttgttkt

SEQ ID NO: 21 - DNA, 57 bp, synthetic construct
  source 1..57: mol_type = other DNA
tccagaaccgccaccgccgctaccgccgccaccgacatttgggaaggactgactctc

SEQ ID NO: 22 - DNA, 53 bp, synthetic construct
  source 1..53: mol_type = other DNA
agcggcggtggcggttctggaggcggcggttctgacattgtgmtgwcacagtc

SEQ ID NO: 23 - DNA, 53 bp, synthetic construct
  source 1..53: mol_type = other DNA
agcggcggtggcggttctggaggcggcggttctgatrttktgatgacccarac

SEQ ID NO: 24 - DNA, 53 bp, synthetic construct
  source 1..53: mol_type = other DNA
agcggcggtggcggttctggaggcggcggttctramattgtgmtgacccaatc

SEQ ID NO: 25 - DNA, 53 bp, synthetic construct
  source 1..53: mol_type = other DNA
agcggcggtggcggttctggaggcggcggttctsaaawtgtkctsacccagtc

SEQ ID NO: 26 - DNA, 53 bp, synthetic construct
  source 1..53: mol_type = other DNA
agcggcggtggcggttctggaggcggcggttctgayatycagatgacmcagwc

SEQ ID NO: 27 - DNA, 50 bp, synthetic construct
  source 1..50: mol_type = other DNA
ctagtggtactccacgcggccgcgtcgacagcmcgtttcagytccarytt

SEQ ID NO: 28 - DNA, 50 bp, synthetic construct
  source 1..50: mol_type = other DNA
ctagtggtactccacgcggccgcgtcgacagcmcgtttkatytccarytt

SEQ ID NO: 29 - DNA, 53 bp, synthetic construct
  source 1..53: mol_type = other DNA
ctagtggtactccacgcggccgcgtcgacagcmcgtttbakytctatctttgt

SEQ ID NO: 30 - DNA, 26 bp, synthetic construct
  source 1..26: mol_type = other DNA
gcagccgctggattgttattactcgc

SEQ ID NO: 31 - DNA, 24 bp, synthetic construct
  source 1..24: mol_type = other DNA
attcagatcctcttctgagatgag